(19) United States
(12) Patent Application Publication
(10) Pub. No.: US 2025/0046437 A1
(43) Pub. Date: Feb. 6, 2025

(54) System and Method for Providing Healthcare Services to Patients

(71) Applicants: Pedram Hamrah, Wellesley, MA (US); Reza Mollaaghababa, Natick, MA (US)

(72) Inventors: Pedram Hamrah, Wellesley, MA (US); Reza Mollaaghababa, Natick, MA (US)

(21) Appl. No.: 18/741,309

(22) Filed: Jun. 12, 2024

Related U.S. Application Data
(63) Continuation of application No. PCT/US2022/052634, filed on Dec. 13, 2022.
(60) Provisional application No. 63/288,829, filed on Dec. 13, 2021.

(51) Int. Cl. G16H 40/20

(52) U.S. Cl. CPC G16H 40/20

(57) ABSTRACT

Embodiments of the innovation relate to, in a healthcare facility server device, a method for providing a personalized healthcare service to a scheduled patient. The method comprises receiving real-time schedule data from a healthcare service provider device, the real-time schedule data identifying a schedule of a healthcare service provider; applying the real-time schedule data to a schedule-monitoring model, the schedule monitoring model configured to predict a future schedule associated with the healthcare service provider; identifying a difference between the real-time schedule data and the future schedule to detect a schedule delay associated with the healthcare service provider; and outputting a delay notification to the scheduled patient of the healthcare service provider, the delay notification associated with the schedule delay.

### RELATED APPLICATIONS

This patent application is a continuation of PCT Application No. PCT/US2022/052634, filed on Dec. 13, 2022, entitled “System and Method for Providing Healthcare Services to Patients,” which claims the benefit of U.S. Provisional Application No. 63/288,829, filed on Dec. 13, 2021, entitled “System and Method for Providing Healthcare Services to Patients,” the contents and teachings of each of which are hereby incorporated by reference in their entirety.

### FIELD

Embodiments of the present innovation relate generally to a system and method for providing healthcare services to patients, and particularly, to such methods and systems that allow for personalized delivery of healthcare services to patients.

### BACKGROUND

Each year, millions of patients across the world visit physicians to seek medical advice regarding a variety of different issues. Typically, such patients schedule an appointment with the physician in advance. On the day of appointment, the patients typically face significant delay in seeing the physician at the scheduled time. Such delays may be caused by unexpected events, or can be due to slight delays associated with each patient, which may cumulatively result in a significant delay for subsequent patients.

### SUMMARY

Conventional healthcare systems can suffer from a variety of deficiencies. For example, conventional healthcare systems do not provide the patients with advanced notice regarding physician schedule delays. Further, conventional healthcare systems do not provide other personalized features, which could facilitate a patient's visit, such as helping the patient to navigate from his/her place of residence to the medical facility at which the medical appointment is scheduled.

By contrast to conventional healthcare systems, embodiments of the present innovation relate to enhanced methods and systems for delivering healthcare services to patients.

In one arrangement, embodiments of the present innovation relate generally to methods and systems for providing personalized healthcare services to patients. By way of example, in some embodiments, the methods and systems according to the present teachings facilitate a patient's visit to a medical facility by providing the patient advanced notification regarding the date/time of the appointment, directions to the facility, and/or real-time information regarding a physician's schedule. Further, the systems and methods of the innovation provide a physician with personal and medical data associated with a patient so as to allow the physician, or other medical professionals, to interact with the patient in a more personalized manner and further to have access to up-to-date patient's medical data.

Embodiments of the innovation relate to, in a healthcare facility server device, a method for providing a personalized healthcare service to a scheduled patient. The method comprises receiving real-time schedule data from a healthcare service provider device, the real-time schedule data identifying a schedule of a healthcare service provider; applying the real-time schedule data to a schedule-monitoring model, the schedule monitoring model configured to predict a future schedule associated with the healthcare service provider; identifying a difference between the real-time schedule data and the future schedule to detect a schedule delay associated with the healthcare service provider; and outputting a delay notification to a scheduled patient of the healthcare service provider, the delay notification associated with the schedule delay.

### BRIEF DESCRIPTION OF THE DRAWINGS

The foregoing and other objects, features and advantages will be apparent from the following description of particular embodiments of the innovation, as illustrated in the accompanying drawings in which like reference characters refer to the same parts throughout the different views. The drawings are not necessarily to scale, emphasis instead being placed upon illustrating the principles of various embodiments of the innovation.

FIG. 1 illustrates a schematic depiction of a system according to an embodiment of the present teachings.

FIG. 2 illustrates a schematic representation of a patient device, according to one arrangement.

FIG. 3 illustrates a schematic representation of a system, according to one arrangement.

### DETAILED DESCRIPTION

Embodiments of the present innovation relate generally to methods and systems for providing personalized healthcare services to patients. By way of example, in some embodiments, the methods and systems according to the present teachings facilitate a patient's visit to a medical facility by providing the patient advanced notification regarding the date/time of the appointment, directions to the facility, and/or real-time information regarding a physician's schedule. Further, the systems and methods of the innovation provide a physician with personal and medical data associated with a patient so as to allow the physician, or other medical professionals, to interact with the patient in a more personalized manner and further to have access to up-to-date patient's medical data.

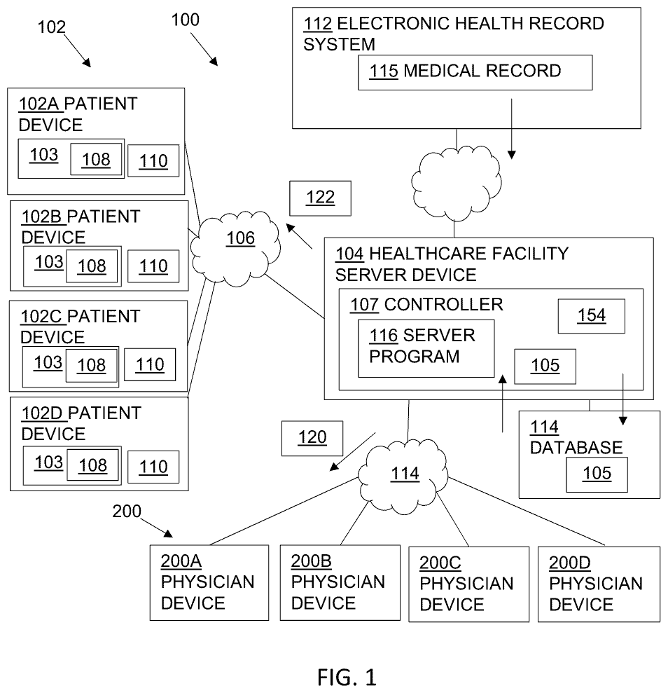

FIG. 1 illustrates a schematic depiction of a system 100 for providing personalized healthcare services to patients, according to an embodiment. The system 100 includes a plurality of patient devices 102a, 102b, 102c, and 102d (herein collectively referred to as devices 102), each of which is associated with an individual, e.g., a patient, seeking medical services. The patient devices 102 can be any suitable device (e.g., a mobile device, such as, a computer, a portable computer, a mobile phone or any other device) capable of communicating with a server device associated with a medical facility and/or an individual medical provider.

The system 100 further includes a healthcare facility server device 104 associated with a medical provider, e.g., a hospital. The healthcare facility server device 104 includes a controller 107, such as a memory and a processor, which is configured to exchange information with the patient devices 102. For example, the healthcare facility server device 104 is disposed in electrical communication with the patient devices 102 via a messaging network 106, e.g., the Internet. In this embodiment, each of the client devices 102 can include a controller 103, such as a processor and memory, which is configured to execute a client program 108 according to the present teachings. As provided in more detail below, when executing the client program 108, the patient devices 102 can obtain information from a patient and communicate that information to the healthcare facility server device 104.

A variety of types of information can be exchanged between a patient device 102 and the healthcare facility server device 104.

In one arrangement, the patient device 102 can provide patient information 105 to the healthcare facility server device 104, such as for use for a medical appointment. For example, when executing the client program 108, the client device 102 can present a questionnaire 110 to a patient to obtain information about the patient. For example, the questionnaire 110 can request information about the patient's marital status, number of children, name, and medical history. The patient can opt not to answer to any of the questions. However, in response to receiving input from the patient, the client device 102 is configured to send the information provided by the patient as patient information 105, such as via a communication module, to the healthcare facility server device 104. The server device 104, in turn, receives the patient information 105, such as via a communication module, and can store this patient information 105 in a database 114 such that the information 105 is associated with that particular patient, such as via a name or medical identification number. As provided in more detail below, the healthcare facility server device 104 can then provide the patient information 105 to a medical provider, such as a physician, upon request.

In one arrangement, the healthcare facility server device 104 can provide notification 122 to a patient device 102, such as for use by a patient for a medical appointment. For example, the controller 107 of the healthcare facility server device 104 can be configured to execute a server program 116 to communicate with a client program 108 of a patient device 102 to provide one or more notifications 122 to the patient regarding an upcoming medical appointment. For example, when executing the server program 116, the healthcare facility server device 104 can provide notification 122 to a patient device 102 regarding an upcoming medical appointment at a plurality of times prior to the appointment (e.g., a month, two weeks, or a week prior to the appointment).

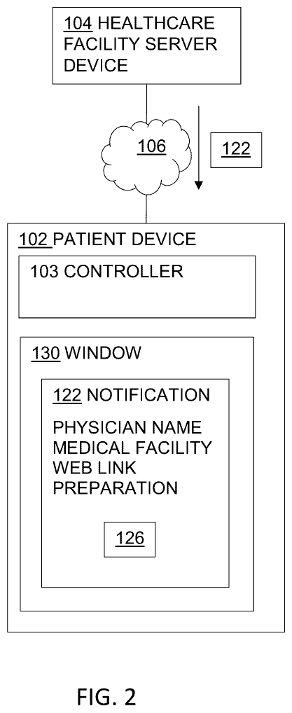

With reference to FIG. 2, and by way of example, the healthcare facility server device 104 can provide such a notification 122 to the patient via a pop-up window 130 appearing on the patient's device 102. Further, in some embodiments, the notification 122 can provide information regarding the medical professional, e.g., a physician, with whom the patient has an appointment.

The notification 122 can include a variety of types of information. By way of example, as shown schematically in FIG. 2, the notification 122 can provide the name of the physician, the medical facility at which the appointment is scheduled, and a web link to the physician's profile. Further, in some embodiments, the notification 122 can remind the patient of any test, or preparation that the patient needs to perform prior to the appointment. By way of illustration, as shown in FIG. 2, when the patient has an appointment for a colonoscopy procedure, the notification 122 can remind the patient of the required preparation for the colonoscopy procedure.

In some embodiments, the notification 122 can further provide directions to the medical facility, including the best route available to the medical facility on the day of the appointment. By way of example, as shown schematically in FIG. 2, on the day of appointment, the patient can launch the notification 122, e.g., via selection of an icon 126. The notification 122 can then provide the time of the appointment and the name of the medical facility to the patient. In addition, the notification 122 can provide the patient with the option of obtaining directions, e.g., in real time, to the medical facility. For example, the patient can choose a MAP icon included with the notification 122 to obtain direction instructions for traveling to the medical facility.

Returning to FIG. 1, the healthcare facility server device 104 can communicate with additional components within the system 100. For example, the healthcare facility server device 104 is disposed in electrical communication with a plurality of computerized devices 200a, 200b, 200c, and 200d (herein referred to as “healthcare workers” or “physician devices” 200) via a messaging network 114, e.g., the Internet, that can be used by medical professionals, e.g., physicians. The physician devices 200 can be any suitable computerized device having a controller (e.g., a memory and processor), such as a portable computer, a mobile phone, or any other suitable device.

The healthcare facility server device 104 can also be disposed in electrical communication with an Electronic Health Record (EHR) system 112 of a medical facility. In one arrangement, the server device 104 is configured to retrieve a patient's medical records 115 (e.g., laboratory tests, X-rays, etc.), from the EHR system 112 and provide that information to one or more of the physician devices 200. In some embodiments, the healthcare facility server device 104 is configured to collate the patient information 105 obtained from a patient device 102 and the patient's medical records 115 received from the EHR system 112 and provide the collated records 120 to a physician device 200 for use by a medical provider, as provided in more detail below.

In certain cases, healthcare workers of a healthcare facility can experience delays in their schedules, which can, in turn, delay the times at which patients are scheduled to see the healthcare workers. To identify the presence of a delay in a healthcare worker's schedule and to provide patients with advanced notice regarding such delays, the controller 107 of the healthcare facility server device 104 can generate a schedule-monitoring model 140.

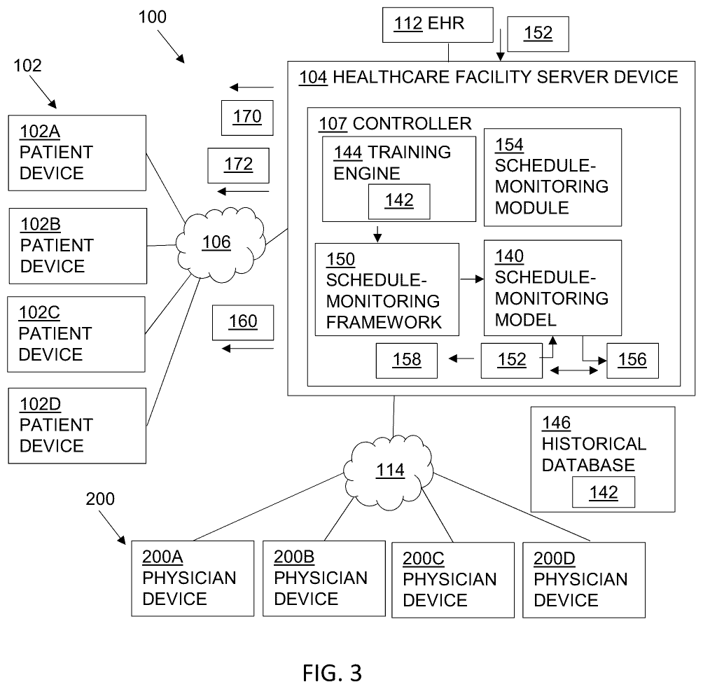

For example, with reference to FIG. 3, with execution of the schedule-monitoring module 154, the healthcare facility server device 104 is configured to generate a schedule-monitoring model 140 based upon historical schedule data 142 such as generated by one or more physician practices associated with a healthcare service provider. For example, the controller 107 can include, and be configured to execute, a training engine 144 on historical schedule data 142 to train a schedule-monitoring framework 150, such as an algorithm, using supervised and unsupervised predictive models to generate the schedule-monitoring model 140.

During a training operation, the training engine 144 can receive the historical schedule data 142 from a historical database 146. While the historical schedule data 142 can be configured in a variety of ways, in one arrangement, the historical schedule data 142 can identify historical schedules for healthcare workers of a particular facility during a given time period, such as over the course of one year. For example, the historical schedule data 142 can identify healthcare workers' schedules by time-of-the-day, by day, by week, or by month. The historical schedule data 142 can identify external schedule factors as well, such as holiday and weather information.

Following receipt of the historical schedule data 142, the training engine 144 can cause the schedule-monitoring framework 150 to perform its operations relative to the historical data 142 to generate the schedule-monitoring model 140. The training engine 144 can generate a variety of schedule-monitoring models 140. In one arrangement, the training engine 144 is configured to generate a schedule-monitoring model 140 for each individual healthcare worker of a given healthcare facility.

For example, the schedule-monitoring framework 150 can be trained to generate a schedule-monitoring model 140 that is configured to estimate the delay time associated with the scheduled times for patients who seek to be examined by a particular medical professional, e.g., a particular physician. In certain cases, the historical schedule data 142 can identify a pattern of delays associated with scheduled appointment times for that medical professional based on data obtained over a given time period, e.g., a month, a few months, a year or multiple years. As such, the schedule-monitoring framework 150 can be trained on that data 142 to generate a schedule-monitoring model 140 which is configured to predict or estimate the delay time, if any, associated with scheduled appointment times for patients. As such, each schedule monitoring module 140 can be configured to predict a particular worker's schedule.

In one arrangement, the training engine 144 is configured to generate a schedule-monitoring model 140 for healthcare workers of a particular department in the facility. As such, the resulting schedule monitoring module 140 is configured to predict the schedule of any or all healthcare workers of a particular department.

Following generation of the schedule-monitoring model 140, the training engine 124 provides the schedule-monitoring model 140 to the healthcare facility server device 104, which can then execute the schedule-monitoring model 140 on real-time healthcare worker schedule data to predict a healthcare worker's schedule.

In one arrangement, during operation and with continued reference to FIG. 3, the healthcare facility server device 104 is configured to receive real-time schedule data 152 from a healthcare service provider device 200 where the real-time schedule data 152 identifies a schedule of a healthcare service provider.

For example, the healthcare facility server device 104 can include a schedule-monitoring module 154 that can track the progress of a physician as the physician attends to patients and can provide notification to one or more patients having scheduled appointments to see that physician regarding whether or not the patient's visit with the physician is likely to be delayed, if so, provide the patient with an approximate delay time.

The schedule-monitoring module 154 can obtain real-time schedule data 152 regarding a physician's daily schedule in a variety of ways. In one arrangement, at the beginning of each working day, the schedule-monitoring module 154 can communicate, via a communication module of the healthcare facility server device 104, with the EHR system 112 to request and obtain a physician's patients schedule for that day. For example, assume the case where Dr. Jones is scheduled to examine eight patients on a given day at the given times. In some cases, the EHR system 112 can receive this information, e.g., in a tabular format, from the physician via the physician device 200. As each patient is examined by the physician and checks out, the EHR system 112 can receive notification and can provide a time stamp to the schedule-monitoring module 154 as the real-time schedule data 152 which corresponds to the patient's check-out time.

Next, the healthcare facility server device 104 is configured to apply the real-time schedule data 152 to the schedule-monitoring model 140 where the schedule monitoring model 140 is configured to predict a future schedule associated with the healthcare service provider.

In one arrangement, with receipt of the real-time schedule data 152, the schedule-monitoring model 140 is configured to calculate the approximate time at which the physician can see the subsequent patients and to determine approximate delays for patients scheduled to visit the physician. For example, as discussed in more detail below, the schedule-monitoring model 140 can operate on the data 152 obtained from the EHR system 112 for that day and/or previous days regarding the check-in/check-out times of the patients to predict the upcoming or future schedule 156, for the physician. In some embodiments, the schedule-monitoring model 140 can further take into account other factors, such as historical data for a particular physician, the day of the week, the month of the year, etc., when predicting the upcoming or future schedule 156 of the healthcare service provider.

If the schedule-monitoring model 140 identifies a difference between the real-time schedule data 152 and the predicted future schedule 156, the schedule-monitoring model 140 can detect a schedule delay 158 associated with the healthcare service provider. For example, in the case where the schedule-monitoring model 140 predicts a future schedule 156 which is delayed relative to a particular patient's actual scheduled appointment, the healthcare facility server device 104 can communicate via a communication module to an associated patient device 102 to provide a schedule discrepancy or delay notification 160 to the patient device 102 regarding the delay in the physician's schedule.

In some embodiments, the schedule-monitoring model 140 can detect the approximate delay times in a physician's schedule at pre-defined times, for example, about two hours prior to each scheduled appointment time for that physician. In some such embodiments, the time at which the detection is made relative to the scheduled appointment time can be informed by the profile of the patient having the scheduled appointment. For example, the schedule-monitoring model 140 can perform the calculation at a time informed by the distance the patient must travel to arrive at the medical facility. In some such embodiments, the schedule-monitoring model 140 can receive data corresponding to the patient's profile, e.g., from the EHR system 112 or by analyzing data provided by the patient in a questionnaire that was previously provided to the patient.

For example, assuming that a patient has an appointment with a physician at 10 am, and the physician is about 1 hour behind schedule, the schedule-monitoring model 140 can communicate the delay notification 160 to the client device 102 to indicate the delay time, and the client device 102 can, in turn, present this information to the patient. For example, the client device 102 can provide a pop-up window to indicate that the physician, e.g., Dr. Jones in this case, is one hour behind schedule. This allows the patient to adjust his or her time of departure, e.g., from home, for traveling to the medical facility. Thus, the patient can avoid unnecessary delay in seeing the physician at the medical facility, and hence can enhance the patient's experience.

In one arrangement, following the detection or prediction of a schedule delay 158, the healthcare facility server device 104 is configured to generate an appointment estimate time 170 which identifies when the healthcare service provider can see the scheduled patient. A variety of methods can be used to generate the appointment estimate time 170 relative to the patient's scheduled appointment time. For example, the estimate 170 can be obtained by analysis of the time a patient is seen by a doctor (herein referred to as “stamp-in time”) relative to the scheduled appointment time for that patient and/or the time at which the patient checks out of the medical facility (herein referred to as “stamp-out time”). For example, healthcare facility server device 104 can perform a regression analysis of the difference between the stamp-in times and the scheduled times of a plurality of patients who have been seen by a physician, or other medical professional, and have checked out of the medical facility can be used to estimate stamp-in times for one or more subsequent patients.

In some embodiments, if the stamp-out time associated with a patient for whom a stamp-in time has been recorded exceeds a predefined threshold, the healthcare facility server device 104 may not consider that patient in the estimation of the stamp-in times for one or more subsequent patients. For example, in some cases, a physician may order certain diagnostic tests for a patient, e.g., an MRI or x-rays, to be performed while the patient is at the medical facility. Meanwhile, the physician can continue examining other patients. The performance of the tests may significantly delay the stamp-out time associated with that patient without affecting the stamp-in times associated with subsequent patients. As such, in such embodiments, the stamp-out time is associated with that patient.

Further, in some cases, the physician may have to attend to an emergency. In such a case, the healthcare facility server device 104 can be provided with this information, e.g., via the EHR system 112, as an emergency notification. Alternatively, the information regarding the emergency can be manually inputted into the healthcare facility server device 104. In some cases, the healthcare facility server device 104 can be provided with an approximate time interval during which the physician may be occupied with attending to the emergency. Alternatively, the healthcare facility server device 104 can be configured to execute an algorithm to predict the amount of time needed by the physician to attend to the emergency.

In one arrangement, following generation of the estimation of the time 170 when the healthcare worker can see the patient, the healthcare facility server device 104 can forward the appointment estimate time 170 to the patient device 102 and can output a travel notification 172 to a patient device 102 to inform the patient scheduled to be examined by the physician regarding the delay in the physician's schedule as well as an estimate as to the travel time from the patient's location to the healthcare facility. By way of example, in some embodiments, the patient's information or profile 105 can include information regarding the patient's residential address. In some such embodiments, the schedule-monitoring model 140 can calculate the approximate time required for the patient to travel from her residential address to the medical facility. The approximate travel time can be obtained, for example, by considering the residential address of the patient, the address of the medical facility and the traffic pattern at the time of the calculation. By way of example, the schedule-monitoring model 140 can obtain the traffic pattern from a source, e.g., a commercially available program such as Waze. Alternatively, an average traffic pattern based on the previously-obtained traffic pattern data for that particular day of the week and the appointment time can be utilized. Such a travel time estimation can be made, for example, at the beginning of each working day, or at a fixed time (e.g., 2-3 hours) prior to each patient's scheduled appointment time.

In one arrangement, the system 200 can be further utilized to improve a patient's experience with a healthcare facility. For example, a physician's device 200 can communicate with the healthcare facility server device 104 to obtain information about the patient. The physician can use this information during the patient's visit to provide more personalized service to the patient. For example, the physician's device 200 can communicate with the healthcare facility server device 104 to receive information regarding a patient collected by the healthcare facility server device 104.

The physician or healthcare worker can obtain a variety of types of information regarding the patient. For example, the physician, or other medical professional, can obtain information about the marital status of a patient, whether or not the patient has children, and if so, the names and birthdates of those children. Further, the physician can obtain other information about the patient's children and/or the patient's spouse, such as their occupation and/or any chronic diseases from which they may suffer. Such information can allow the physician, or other medical professional, to provide a more personalized treatment to the patient. In other words, such information can allow the physician, or other medical professional, to interact with a patient in a manner that will lead to enhanced satisfaction of the patient and a better treatment outcome.

As provided above the healthcare facility server device 104 can provide a travel notification 172 to a patient device 102 to inform a patient of a delay in the physician's schedule. In one arrangement, the healthcare facility server device 104 can review additional variables prior to sending the travel notification 172. For example, the healthcare facility server device 104 can determine how far the patient is living (e.g., in state vs. out of state) or prior visits of patient (e.g., history of the patient's visits) prior to transmitting the travel notification 160.

In one arrangement, each patient can utilize the patient device 102 to track where the patient is in the schedule and how the patient is advancing in the schedule.

In one arrangement, a patient's chart can include barcodes which identify scheduling information pertaining to a patient's visit at a healthcare facility. For example, the barcodes can identify the following times associated with a patient's visit: patient arriving; time chart is pulled by technician; time patient is seen by physician; time when the leave the physician; and time of patient checking out.

As provided above, the patient device 102 can provide patient information 105 to the healthcare facility server device 104. In one arrangement, the patient information 105 is configured as a brief synopsis of the patient's data or as a personalization of the patient's information to be presented to the physician. The healthcare facility server device 104 can update exiting patient information with the received patient information 105 (e.g., nothing has changed, somebody has to update it, if needed, etc.).

As provided above, the client devices 102 can execute a client program 108 in order to obtain information from a patient and communicate that information to the healthcare facility server device 104. In one arrangement, the client program 108 is further configured to notify the patient if the healthcare appointment is one-time only or if additional appointments have been scheduled.

While various embodiments of the innovation have been particularly shown and described, it will be understood by those skilled in the art that various changes in form and details may be made therein without departing from the spirit and scope of the innovation as defined by the appended claims.

What is claimed is:

1. In a healthcare facility server device, a method for providing a personalized healthcare service to a scheduled patient, comprising:
receiving real-time schedule data from a healthcare service provider device, the real-time schedule data identifying a schedule of a healthcare service provider;
applying the real-time schedule data to a schedule-monitoring model, the schedule monitoring model configured to predict a future schedule associated with the healthcare service provider;
identifying a difference between the real-time schedule data and the future schedule to detect a schedule delay associated with the healthcare service provider; and
outputting a delay notification to the scheduled patient of the healthcare service provider, the delay notification associated with the schedule delay.

2. The method of claim 1, wherein detecting the schedule delay associated with the healthcare service provider further comprises:
generating an appointment estimate time which identifies when the healthcare service provider can meet with the scheduled patient; and
outputting the appointment estimate time to the scheduled patient.

3. The method of claim 2, comprising outputting a travel notification to the scheduled patient, the travel notification providing an estimate regarding a travel time between the scheduled patient's location and healthcare service provider's facility.

4. A healthcare facility server device, comprising a controller having a memory and a processor, the controller configured to:
receive real-time schedule data from a healthcare service provider device, the real-time schedule data identifying a schedule of a healthcare service provider;
apply the real-time schedule data to a schedule-monitoring model, the schedule monitoring model configured to predict a future schedule associated with the healthcare service provider;
identify a difference between the real-time schedule data and the future schedule to detect a schedule delay associated with the healthcare service provider; and
output a delay notification to the scheduled patient of the healthcare service provider, the delay notification associated with the schedule delay.

5. The healthcare facility server device of claim 4, wherein when detecting the schedule delay associated with the healthcare service provider, the controller is further configured to:
generate an appointment estimate time which identifies when the healthcare service provider can meet with the scheduled patient; and
output the appointment estimate time to the scheduled patient.

6. The healthcare facility server device of claim 5, wherein the controller is configured to output a travel notification to the scheduled patient, the travel notification providing an estimate regarding a travel time between the scheduled patient's location and healthcare service provider's facility.
